(19) United States
(12) Patent Application Publication
(10) Pub. No.: US 2023/0191091 A1
(43) Pub. Date: Jun. 22, 2023

(54) BALLOON CATHETER

(71) Applicant: ASAHI INTECC CO., LTD., Seto-shi (JP)

(72) Inventors: Shota ENDO, Seto-shi (JP); Keiji IIDA, Seto-shi (JP)

(73) Assignee: ASAHI INTECC CO., LTD., Seto-shi (JP)

(21) Appl. No.: 18/158,630

(22) Filed: Jan. 24, 2023

Related U.S. Application Data
(63) Continuation of application No. PCT/JP2020/029448, filed on Jul. 31, 2020.

(51) Int. Cl. A61M 25/10

(52) U.S. Cl. CPC A61M 25/1036; A61M 2025/1075

(57) ABSTRACT

A balloon catheter includes a shaft having a first shaft portion and a second shaft portion located on a proximal side of the first shaft portion, and a balloon covering and joined to the second shaft portion. The first shaft portion is more flexible than the second shaft portion and has a length in an axial direction of 1.5 cm or more.

### CROSS REFERENCE TO RELATED APPLICATIONS

This is a Continuation of PCT/JP2020/029448 filed on Jul. 31, 2020. The disclosure of the prior application is hereby incorporated by reference herein in its entirety.

### TECHNICAL FIELD

The technology disclosed in the present specification relates to a balloon catheter.

### BACKGROUND

A balloon catheter including a balloon can be inserted into a body lumen, such as a blood vessel, and inflated/deflated. The balloon catheter includes a shaft and a balloon that covers a part of the shaft while being joined to the shaft (e.g., refer to Patent literature 1 below). A part of the shaft to which the balloon is joined (hereinafter referred to as a “balloon joint portion”) and a part of the shaft on a further distal side than the balloon (hereinafter referred to as a “catheter distal portion”) have the same flexibility.

### CITATION LIST

### Patent Literature

Patent Literature 1: JP 2007-507305 W

### SUMMARY

### Technical Problem

In the conventional balloon catheter described above, the catheter distal portion has only the same degree of flexibility as the balloon joint portion. As such, when the balloon catheter is inserted into a bent part of the blood vessel, for example, it is difficult to insert the catheter distal portion into the bent part. As a result, the distal end of the balloon catheter cannot be brought close to a lesion (e.g., a stenosis or occluded part) in the blood vessel. In such a case, for example, it becomes difficult to apply an appropriate treatment to the lesion using the balloon catheter.

### Solution to Problem

The present specification discloses a technology capable of solving the above-mentioned problems.

The technology disclosed in the present specification can be implemented, for example, according to the following aspects.

(1) A balloon catheter disclosed in the present specification includes a shaft having a first shaft portion and a second shaft portion located on a proximal side of the first shaft portion, and a balloon covering and joined to the second shaft portion. The first shaft portion is more flexible than the second shaft portion and has a length in an axial direction of 1.5 cm or more. In this balloon catheter, the shaft includes the first shaft portion on a distal side of the second shaft portion to which the balloon is joined. This first shaft portion is more flexible than the second shaft portion and has the length in the axial direction of 1.5 cm or more. According to this balloon catheter, a distal end of the balloon catheter (first shaft portion) can be easily inserted to a vicinity of the lesion as compared with, for example, a configuration without the first shaft portion, making it possible to shorten the distance between the distal end of the balloon catheter and the lesion.

(2) The balloon catheter may be configured such that an outer layer of the first shaft portion and an outer layer of the second shaft portion are each constituted of a resin, and a resin material of the outer layer of the first shaft portion has a Shore hardness of D47 or less. According to this balloon catheter, the distal end of the balloon catheter can be more easily inserted to the vicinity of the lesion as compared with a configuration in which the resin material of the outer layer of the first shaft portion has the Shore hardness of more than D47, making it possible to further shorten the distance between the distal end of the balloon catheter and the lesion.

(3) The balloon catheter may be configured such that the outer layer of the first shaft portion and the outer layer of the second shaft portion are each constituted of the resin, and the resin material of the outer layer of the first shaft portion has the Shore hardness of D40 or less. According to this balloon catheter, the distal end of the balloon catheter can be more easily inserted to the vicinity of the lesion as compared with the configuration in which the resin material of the outer layer of the first shaft portion has the Shore hardness of more than D40, making it possible to further shorten the distance between the distal end of the balloon catheter and the lesion.

(4) The balloon catheter may be configured such that a first lumen penetrating from the proximal side of the second shaft portion to the distal end of the first shaft portion is formed in the shaft, and a side hole communicating with the first lumen is formed in an outer peripheral surface of the first shaft portion on the proximal side. In this balloon catheter, the first shaft portion protrudes further to the distal side than the balloon. For this reason, for example, when a recovery object (e.g., a thrombus piece, etc.) is recovered from the lesion, the recovery object may enter the outer peripheral side of the first shaft portion instead of being recovered into an opening of the first lumen formed at the distal end of the first shaft portion. However, according to this balloon catheter, the unrecovered recovery object can be recovered through the side hole.

(5) The balloon catheter may be configured such that a hydrophilic coating is formed at least on the outer peripheral surface of the first shaft portion on the distal side. According to this balloon catheter, the slidability of the first shaft portion is improved by the hydrophilic coating, making it possible to improve insertability of the first shaft portion into the body lumen.

(6) The balloon catheter may be configured such that the hydrophilic coating is formed on the outer peripheral surface of a distal portion of the first shaft portion, and the coefficient of friction of the outer peripheral surface of the first shaft portion on the further proximal side than the hydrophilic coating is higher than the coefficient of friction of the hydrophilic coating. According to this balloon catheter, the hydrophilic coating having the relatively low coefficient of friction is formed in the distal portion of the first shaft portion, thus the distal side of the first shaft portion can be easily inserted into the body lumen. Further, the coefficient of friction of the outer peripheral surface of the first shaft portion on the further proximal side than the hydrophilic coating is higher than the coefficient of friction of the hydrophilic coating. Thus, the proximal side of the first shaft portion having the relatively high coefficient of friction is restricted in relative movement due to contact with, for example, the inner wall surface in the bent body lumen. This allows the first shaft portion of the balloon catheter to be inserted in the body lumen and can improve a retention state of the inserted catheter.

(7) The balloon catheter may be configured such that the first shaft portion and the second shaft portion are integrally formed, a first lumen and a second lumen are formed in the shaft, the first lumen penetrating from the proximal side of the second shaft portion to the distal end of the first shaft portion, the second lumen extending from the proximal side of the second shaft portion to an inside of the balloon, thereby communicating with the balloon, and an outer diameter of the first shaft portion is smaller than the outer diameter of the second shaft portion. The shafts are integrally formed, the second lumen (expansion lumen) is not formed in the first shaft portion, and the first shaft portion has the smaller diameter than the second shaft portion.

### Advantageous Effects

In this balloon catheter, the first shaft portion and the second shaft portion can be integrally formed. In that case, the torque transmissibility of the shafts is higher as compared with a configuration in which the first shaft portion and the second shaft portion are formed separately and joined to each other. Further, a difference in the flexibility between the first shaft portion and the second shaft portion can be achieved by the difference in the number of the lumens and the outer diameter. That is, according to this balloon catheter, it is possible to shorten the distance between the distal end of the balloon catheter and the lesion while preventing a reduction in the torque transmissibility of the shafts.

### BRIEF DESCRIPTION OF DRAWINGS

FIG. 1 is an explanatory diagram schematically illustrating a configuration of a balloon catheter 100 in a first embodiment.

FIG. 2 is an explanatory diagram (cross-sectional view) schematically illustrating a distal shaft portion 14 of a shaft 10.

FIG. 3 is an explanatory diagram (cross-sectional view) schematically illustrating a proximal shaft portion 16 of the shaft 10.

FIG. 4 is an explanatory diagram illustrating a state in which the balloon catheter 100 is placed in a blood vessel.

FIG. 5 is an explanatory diagram schematically illustrating a configuration of a balloon catheter 100a in a second embodiment.

FIG. 6 is an explanatory diagram (cross-sectional view) schematically illustrating a proximal shaft portion 16a of a shaft 10a.

FIG. 7 is an explanatory diagram illustrating a state in which the balloon catheter 100a is placed in the blood vessel.

FIGS. 8A and 8B are explanatory diagrams illustrating how a thrombus piece C is recovered by side holes 20.

### DETAILED DESCRIPTION OF EMBODIMENTS

### A-1. Basic Configuration of Balloon Catheter 100:

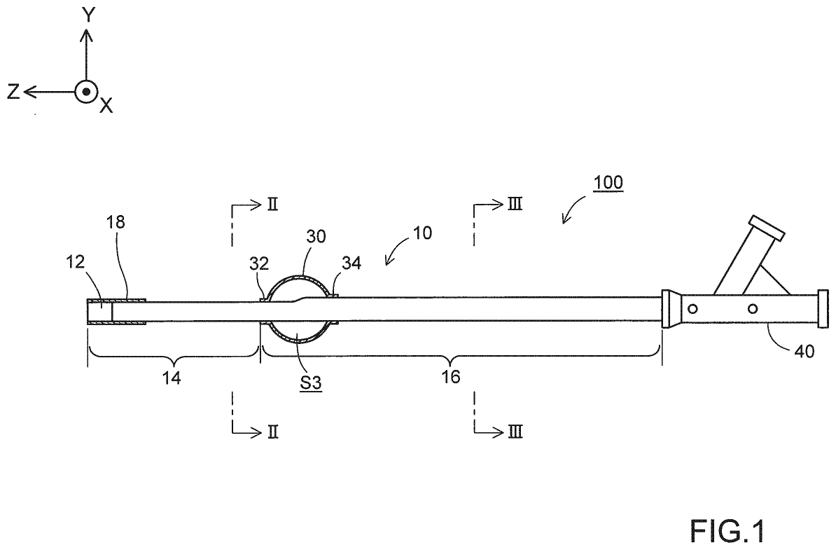

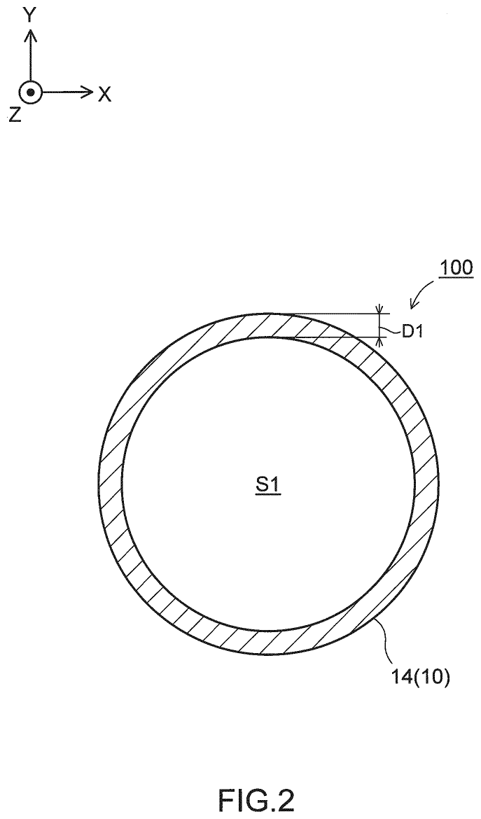

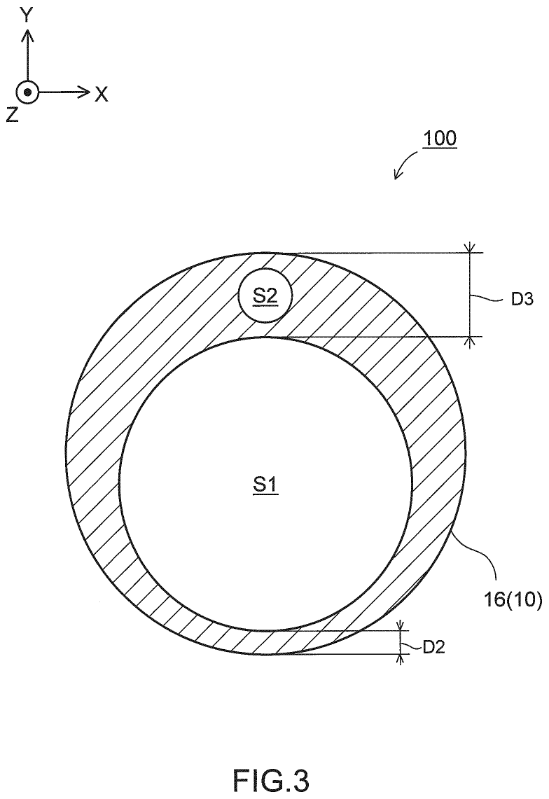

FIG. 1 is an explanatory diagram schematically illustrating a configuration of a balloon catheter 100 in a first embodiment. FIG. 2 is an explanatory diagram schematically illustrating a distal shaft portion 14 of a shaft 10. FIG. 3 is an explanatory diagram schematically illustrating a proximal shaft portion 16 of the shaft 10. FIG. 2 shows a configuration of the shaft 10 (distal shaft portion 14) in a cross section (XY section: cross-sectional view obtained by cutting along a plane including the x-axis and the y-axis shown in FIG. 1) at a position II-II in FIG. 1. FIG. 3 shows a configuration of the shaft 10 (proximal shaft portion 16) in a cross section at a position in FIG. 1. Note that, in FIG. 2 and FIG. 3, a balloon 30 and a connector 40, which will be described later, are omitted.

In FIG. 1, a positive direction side of the Z axis (side of a distal tip 12 of the balloon catheter 100) is the tip end side (distal side) to be inserted into the body, and a negative direction side of the Z axis (side opposite to the side of the distal tip 12 of the balloon catheter 100) is the base end side (proximal side) to be operated by an operator such as a doctor. Note that, although FIG. 1 shows that the balloon catheter 100 has, as a whole, a linear shape parallel to the Z-axis direction, the balloon catheter 100 is flexible enough to be bent. Further, FIG. 1 shows a state in which the balloon 30 described below is inflated.

The balloon catheter 100 is a medical device which is inserted into the blood vessel, for example, in order to block a blood flow by inflating the balloon 30 so as to be in close contact with the blood vessel wall on a front side of a lesion (a stenosis or occluded part) in the blood vessel. As shown in FIG. 1, the balloon catheter 100 includes the shaft 10 and the balloon 30.

The shaft 10 is a tubular (e.g., cylindrical) member with distal and proximal ends opened. Note that, in the present specification, the term “tubular (cylindrical)” is not limited to a complete tubular shape (cylindrical shape) and may include a substantially tubular shape (substantially cylindrical shape, e.g., a slightly conical shape, a shape with some unevenness, and the like) as a whole. As shown in FIG. 2 and FIG. 3, a main lumen S1 and an expansion lumen S2 are formed inside the shaft 10. A linear device (not shown) such as a guide wire, a retrieval device (stent retriever, aspiration catheter), or a dilator can be inserted through the main lumen S1. An inflation fluid for inflating the balloon 30 can flow through the expansion lumen S2. Note that the fluid may be gas or liquid, and examples thereof include gas such as helium gas, CO₂ gas, or O₂ gas, and liquid such as physiological saline or a contrast medium. That is, the balloon catheter 100 is a so-called two-lumen type catheter including the main lumen S1 and the expansion lumen S2. The main lumen S1 is an example of a first lumen in the scope of claims, and the expansion lumen S2 is an example of a second lumen in the scope of claims.

Specifically, the shaft 10 includes the distal shaft portion 14 and the proximal shaft portion 16. The distal shaft portion 14 is an example of a first shaft portion in the scope of claims, and the proximal shaft portion 16 is an example of a second shaft portion in the scope of claims.

The distal shaft portion 14 is a portion that includes the distal end of the shaft 10. The distal shaft portion 14 has a single lumen structure in which only the main lumen Si is formed. The main lumen S1 penetrates the entire length of the distal shaft portion 14 in the axial direction. As shown in FIG. 2, when viewed from the axial direction of the balloon catheter 100 (Z-axis direction in each drawing), the main lumen S1 is formed substantially in the central part of the distal shaft portion 14. When viewed from the axial direction, a thickness D1 of the distal shaft portion 14 is substantially the same over the entire periphery. Note that, in the present embodiment, when viewed from the axial direction, both the outer peripheral shape and the inner peripheral shape of the distal shaft portion 14 are substantially circular shape. The length of the distal shaft portion 14 in the axial direction can be, for example, 2 cm.

Note that the distal tip 12 is disposed at the distal end of the distal shaft portion 14. The distal tip 12 is a tubular member with front and rear ends opened. The distal tip 12 may have an outer shape with the same outer diameter over the entire length or may have a tapered outer shape in which the outer diameter gradually decreases toward the distal end. A device inserted into the main lumen S1 is led out from the distal end of the distal tip 12 to the outside. The distal tip 12 has flexibility equal to or more than that of the proximal side of the distal shaft portion 14.

The proximal shaft portion 16 is a portion that includes the proximal end of the shaft 10. The distal shaft portion 14 and the proximal shaft portion 16 are adjacent to each other in the axial direction. The proximal shaft portion 16 has a double lumen structure in which the expansion lumen S2 is formed in addition to the main lumen S1. The main lumen S1 penetrates the entire length of the proximal shaft portion 16 in the axial direction. The expansion lumen S2 extends from the proximal end of the proximal shaft portion 16 toward the distal side and opens to the outer peripheral surface of the distal portion of the proximal shaft portion 16. Specifically, as shown in FIG. 3, when viewed from the axial direction, the main lumen S1 is formed at a slightly eccentric position in the proximal shaft portion 16, and the expansion lumen S2 is formed on the outer peripheral side of the main lumen S1. The diameter of the expansion lumen S2 is smaller than the diameter of the main lumen S1.

The diameter of the main lumen S1 formed in the distal shaft portion 14 and the diameter of the main lumen S1 formed in the proximal shaft portion 16 are substantially the same. Note that, in the present specification, the phrase “A and B being substantially the same” means that the error between A and B is 5% or less of A or B. That is, the main lumen S1 having substantially the same diameter is formed so as to penetrate the entire length of the shaft 10. When viewed from the axial direction, a thickness D2 of the proximal shaft portion 16 on the side opposite the expansion lumen S2 is substantially the same as the thickness D1 of the distal shaft portion 14. A thickness D3 of the proximal shaft portion 16 on the side where the expansion lumen S2 is formed is larger than the thickness D2 on the side opposite to the expansion lumen S2. In this manner, the distal shaft portion 14 has a thinner part as compared with the proximal shaft portion 16, and the outer diameter of the distal shaft portion 14 is smaller than the outer diameter of the proximal shaft portion 16. This makes the distal shaft portion 14 more flexible than the proximal shaft portion 16. For example, the Shore hardness of an outer layer resin material of the distal shaft portion 14 is D47 or less. The Shore hardness of the outer layer resin material of the distal shaft portion 14 may be D40 or less. Note that the connector 40 is attached to the proximal end of the proximal shaft portion 16 for introducing a device, a fluid, and the like into the lumens S1 and S2.

In the present embodiment, the shaft 10 is integrally formed of the same material in the entire shaft. The shaft 10 is preferably formed of a material that can be heat-sealed and has a certain degree of flexibility. Examples of the material for forming the shaft 10 include a thermoplastic resin, more specifically a polyolefin such as polyethylene, polypropylene, polybutene, an ethylene-propylene copolymer, an ethylene-vinyl acetate copolymer, an ionomer, or a mixture of two or more kinds thereof, a polyvinyl chloride resin, polyamide, nylon, a polyamide elastomer, polyester, a polyester elastomer, thermoplastic polyurethane, and the like.

As shown in FIG. 1, on the distal portion of the outer peripheral surface of the distal shaft portion 14 of the shaft 10, a hydrophilic coating 18 formed of a hydrophilic resin is formed over the entire periphery of the distal shaft portion 14. The hydrophilic coating 18 is provided to reduce the frictional resistance between the surface of the distal shaft portion 14 and the blood vessel inner wall, thereby ensuring slidability, when the distal shaft portion 14 is inserted into the blood vessel. Note that, in the present embodiment, the hydrophilic coating 18 is not formed on the proximal side of the distal shaft portion 14, making the coefficient of friction of the outer peripheral surface higher than that in the distal portion.

The balloon 30 is an inflation portion that can be inflated and deflated with supply and exhaustion of a fluid. The balloon 30 covers the outer periphery of the distal portion of the proximal shaft portion 16. A distal portion 32 and a rear portion 34 of the balloon 30 are each joined to the outer peripheral surface of the distal portion of the proximal shaft portion 16 by, for example, welding. Note that, in the present embodiment, the distal portion 32 of the balloon 30 is joined to the outer peripheral surface of the distal end of the proximal shaft portion 16, and the joint part between the distal portion 32 and the proximal shaft portion 16 is adjacent to the distal shaft portion 14 in the axial direction. The expansion lumen S2 described above communicates with an internal space S3 (refer to FIG. 1) formed between the balloon 30 and the proximal shaft portion 16. The balloon 30 is folded (not shown) so as to be in close contact with the outer peripheral surface of the proximal shaft portion 16 when it is deflated. Note that the length of the balloon 30 in the axial direction can be, for example, about 2 cm.

The balloon 30 is preferably formed of a material having a certain degree of flexibility, and more preferably formed of a material that is thinner than the shaft 10 and has flexibility. Examples of the material for forming the balloon 30 include a thermoplastic resin such as a polyolefin such as polyethylene, polypropylene, polybutene, an ethylene-propylene copolymer, an ethylene-vinyl acetate copolymer, an ionomer, or a mixture of two or more kinds thereof, a soft polyvinyl chloride resin, polyamide, a polyamide elastomer, polyester, a polyester elastomer, polyurethane, or a fluororesin, silicone rubber, latex rubber, and the like.

### A-2. Usage Example of Balloon Catheter 100:

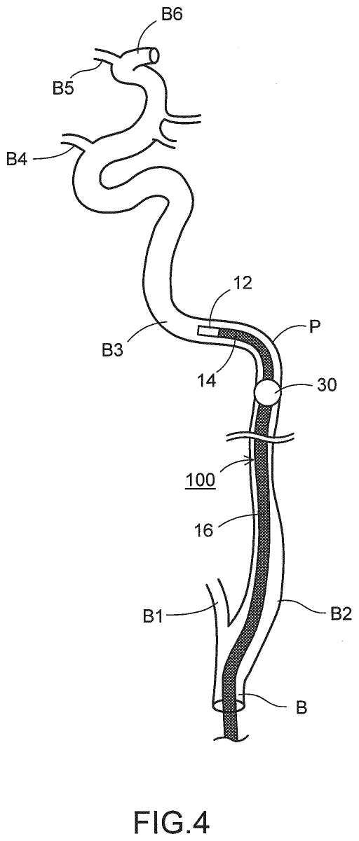

Next, a usage example of the balloon catheter 100 in the first embodiment will be described. FIG. 4 is an explanatory diagram illustrating a state in which the balloon catheter 100 is placed in the blood vessel. FIG. 4 schematically shows the blood vessels on the distal side of the carotid artery (a common carotid artery B, an external carotid artery B1, a cervical part of internal carotid artery B2, a petrous part of internal carotid artery B3, an ophthalmic artery B4, an anterior cerebral artery B5, and a middle cerebral artery B6). The balloon catheter 100 is a balloon-equipped guiding catheter and can be used, for example, for a thrombectomy treatment in acute cerebral infarction.

Specifically, the balloon catheter 100 with the deflated balloon 30 is inserted into the blood vessel and pushed through the common carotid artery B into, for example, the cervical part of internal carotid artery B2. A petrous P, which is a bent blood vessel, exists in the cervical part of internal carotid artery B2. Here, the proximal shaft portion 16 of the shaft 10 has relatively low flexibility (high rigidity), and in particular, the joint part between the balloon 30 and the proximal shaft portion 16 has low flexibility. This makes it difficult to insert the proximal shaft portion 16 into the petrous P.

On the other hand, the distal shaft portion 14 of the shaft 10 has relatively high flexibility, and the length of the distal shaft portion 14 in the axial direction is 1.5 cm or more. Thus, as shown in FIG. 4, the distal shaft portion 14 can be inserted into the petrous P relatively easily. The balloon 30 is inflated while the distal shaft portion 14 is inserted into the petrous P, so that the blood flow in the carotid artery is temporarily blocked. At this time, the balloon 30 is positioned, for example, in front of the petrous P in the cervical part of internal carotid artery B2. Then, a retrieval device (not shown) is led out from the distal end of the balloon catheter 100 through the main lumen S1 and delivered to a vicinity of the lesion existing beyond the petrous P (e.g., the petrous part of internal carotid artery B3 or the like). Next, a thrombus piece is captured using the retrieval device, and the retrieval device having captured the thrombus piece is retracted into the main lumen S1 of the balloon catheter 100. In this manner, the thrombus piece in the blood vessel can be recovered.

### A-3. Advantageous Effects of Present Embodiment:

As described above, in the balloon catheter 100 of the present embodiment, the distal shaft portion 14 having relatively high flexibility is disposed on the distal side of the balloon 30 (refer to FIG. 1 and FIG. 2). As a result, in the present embodiment, the distance between the distal end of the balloon catheter 100 and the lesion in the blood vessel (hereinafter referred to as “thrombus recovery distance”) becomes shorter as compared with a configuration without the distal shaft portion 14. If the thrombus recovery distance is shortened, for example, a damage to the blood vessel or the like caused by the retrieval device can be reduced as the distance the retrieval device travels in the blood vessel in a state of being exposed becomes shorter. For example, it is possible to reduce the risk of having the blood vessel perforation or spasm due to scratching of the blood vessel or the like caused by the retrieval device.

Further, as shown in FIG. 4, the distal shaft portion 14 is bent inside the petrous P, which fixes the position of the distal shaft portion 14 in the blood vessel and improves support force of the balloon catheter 100. That is, in general, the flexibility of the shaft is preferably reduced to improve the support force. On the other hand, the shaft having low flexibility has less insertability (blood vessel followability) in the blood vessel. Regarding this, in the present embodiment, the distal shaft portion 14, which is flexible enough to be bent in the petrous P, is disposed on the distal side of the balloon 30. This can improve the support force of the balloon catheter 100 by bending of the distal shaft portion 14 while ensuring the blood vessel followability of the distal shaft portion 14.

In particular, the hydrophilic coating 18 having the relatively low coefficient of friction is formed in the distal portion of the distal shaft portion 14. This allows the distal side of the distal shaft portion 14 to be smoothly inserted into the petrous P. Further, the hydrophilic coating 18 is not formed on the proximal side of the distal shaft portion 14, providing the relatively high coefficient of friction. As a result, the proximal side of the distal shaft portion 14 is brought into contact with the inner wall surface of the petrous P, while the distal shaft portion 14 is bent, allowing the position of the distal shaft portion 14 in the blood vessel to be more firmly fixed. This can further improve the support force of the balloon catheter 100.

### B-1. Basic Configuration of Balloon Catheter 100a:

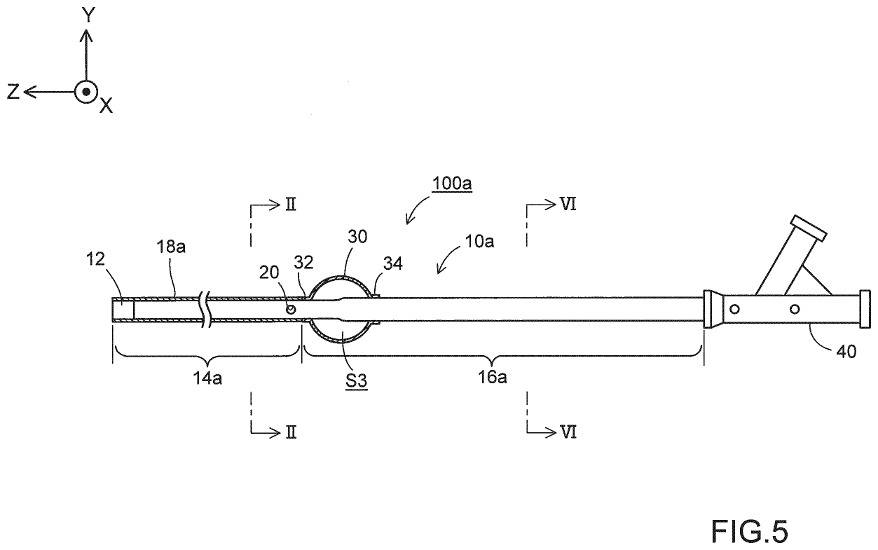

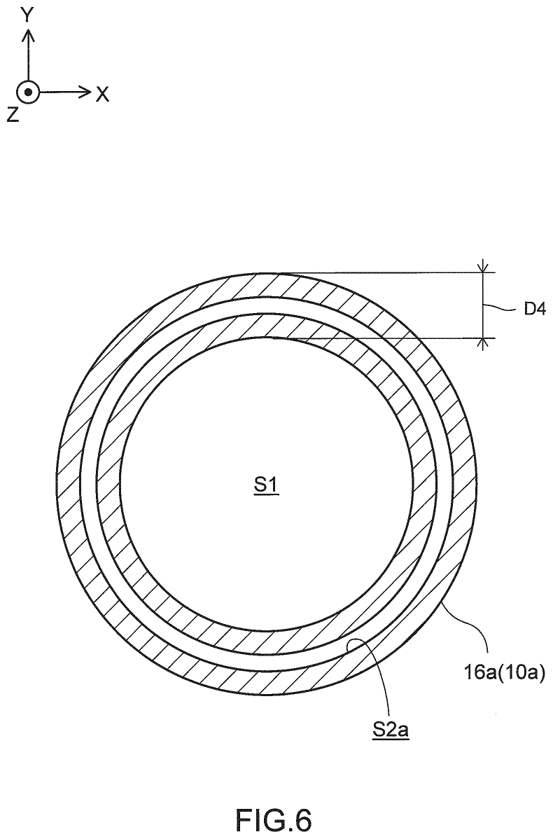

FIG. 5 is an explanatory diagram schematically illustrating a configuration of a balloon catheter 100a in a second embodiment. FIG. 6 is an explanatory diagram schematically illustrating a proximal shaft portion 16a of a shaft 10a. FIG. 6 shows a configuration of the shaft 10a (proximal shaft portion 16a) in a cross section at a position VI-VI in FIG. 5. Note that the balloon 30 and the connector 40 are omitted in FIG. 6. Further, the configuration of the shaft 10a (distal shaft portion 14a) in a cross section at a position II-II in FIG. 6 is the same as in FIG. 2 of the above-mentioned first embodiment. In the following, in the configuration of the balloon catheter 100a of the second embodiment, the same constituents as those of the balloon catheter 100 of the above-mentioned first embodiment are denoted by the same reference signs, and the description thereof will be omitted as appropriate.

The shaft 10a included in the balloon catheter 100a is a tubular (e.g., cylindrical) member with distal and proximal ends opened. As shown in FIG. 2 and FIG. 6, the main lumen S1 and an expansion lumen S2a are formed inside the shaft 10a. The expansion lumen S2a is an example of the second lumen in the scope of claims.

Specifically, the shaft 10a includes the distal shaft portion 14a and the proximal shaft portion 16a. The distal shaft portion 14a is an example of the first shaft portion in the scope of claims, and the proximal shaft portion 16a is an example of the second shaft portion in the scope of claims.

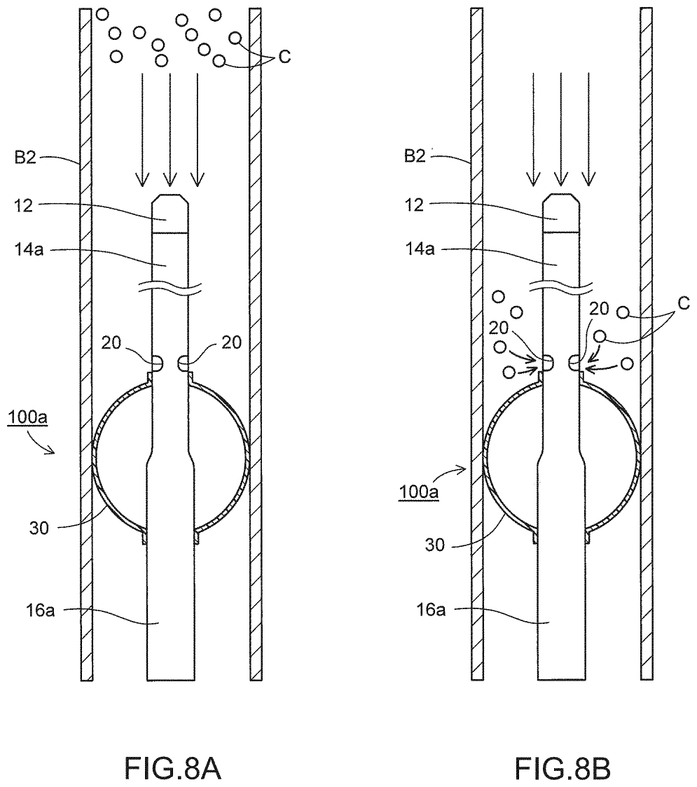

Like the distal shaft portion 14 of the above-mentioned first embodiment, the distal shaft portion 14a has a single lumen structure in which only the main lumen S1 is formed (refer to FIG. 2). However, the length of the distal shaft portion 14a in the axial direction is longer than the length of the distal shaft portion 14 in the axial direction of the above-mentioned first embodiment, and it can be, for example, 20 cm. Further, a side hole 20 communicating with the main lumen S1 is formed in the outer peripheral surface of the distal shaft portion 14a on the proximal side. In the present embodiment, a pair of the side holes 20 are disposed at mutually symmetrical positions with respect to the central axis of the distal shaft portion 14a (refer to FIGS. 8A and 8B below).

The proximal shaft portion 16a has a coaxial structure in which the expansion lumen S2a is formed in addition to the main lumen S1. The expansion lumen S2a extends from the proximal end of the proximal shaft portion 16a toward the distal side and opens to the outer peripheral surface of the distal portion of the proximal shaft portion 16a. Specifically, as shown in FIG. 6, when viewed from the axial direction, the main lumen S1 is formed in the central part in the proximal shaft portion 16a, and the expansion lumen S2a is annularly formed so as to surround the outer periphery of the main lumen S1. The diameter of the main lumen S1 formed in the distal shaft portion 14a and the diameter of the main lumen S1 formed in the proximal shaft portion 16a are substantially the same. That is, the main lumen S1 having substantially the same diameter is formed so as to penetrate the entire length of the shaft 10a. The thickness D1 of the distal shaft portion 14a is smaller than a thickness D4 of the proximal shaft portion 16a, and the outer diameter of the distal shaft portion 14a is smaller than the outer diameter of the proximal shaft portion 16a. This makes the distal shaft portion 14a more flexible than the proximal shaft portion 16a. For example, the Shore hardness of an outer layer resin material of the distal shaft portion 14a is D47 or less. The Shore hardness of the outer layer resin material of the distal shaft portion 14a may be D40 or less.

As shown in FIG. 5, a hydrophilic coating 18a formed of a hydrophilic resin is formed on the entire outer peripheral surface of the distal shaft portion 14a of the shaft 10a.

### B-2. Usage Example of Balloon Catheter 100a:

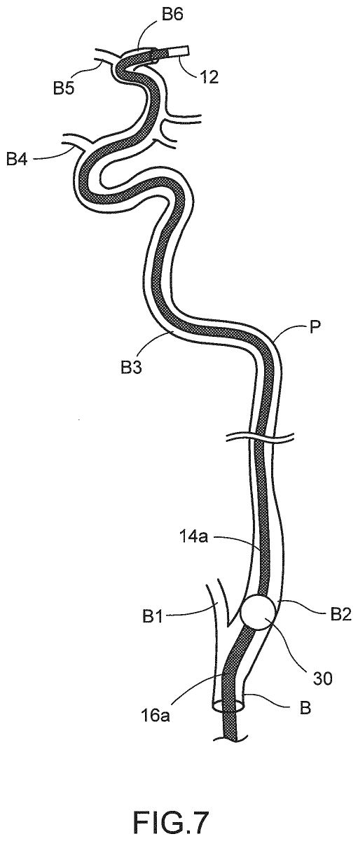

Next, a usage example of the balloon catheter 100a in the second embodiment will be described. FIG. 7 is an explanatory diagram illustrating a state in which the balloon catheter 100a is placed in the blood vessel. As shown in FIG. 7, the balloon 30 is positioned near the entrance of the cervical part of internal carotid artery B2, and the distal shaft portion 14a extends beyond the petrous P to near the middle cerebral artery B6. As described above, when the length of the distal shaft portion 14a in the axial direction is about 20 cm, the distal end of the balloon catheter 100a can be brought closer to the vicinity of the lesion located further beyond the petrous P.

FIGS. 8A and 8B are explanatory diagrams illustrating how a thrombus piece C is recovered by the side holes 20. In the balloon catheter 100a, the distal shaft portion 14a protrudes further to the distal side than the balloon 30. For this reason, for example, when the thrombus piece C is recovered from the lesion, the thrombus piece C may not be recovered in the opening of the main lumen S1 formed at the distal end of distal shaft portion 14a, but instead enter the outer peripheral side of the distal shaft portion 14a (refer to FIGS. 8A and 8B). However, according to the present embodiment, the unrecovered thrombus piece C is recovered into the main lumen S1 through the side holes 20 formed in the distal shaft portion 14a.

### C. Modifications:

The technology disclosed in the present specification is not limited to the above-described embodiments and can be modified in various forms without departing from the scope of the disclosed embodiments. For example, the following modifications are possible.

The configurations of the balloon catheters 100 and 100a in the above-mentioned embodiments are merely examples, and various modifications are possible. For example, the length of the distal shaft portions 14 and 14a in the axial direction in each of the above-mentioned embodiments may be any length of 1.5 cm or more. Specifically, in the above-mentioned first embodiment, the length of the distal shaft portion 14 in the axial direction may be 1.5 cm or more and less than 2 cm, or longer than 2 cm. In the above-mentioned second embodiment, the length of the distal shaft portion 14a in the axial direction may be 1.5 cm or more and less than 20 cm, or longer than 20 cm. In each of the above-mentioned embodiments, the distal shaft portions 14 and 14a may be configured without the distal tip 12.

In each of the above-mentioned embodiments, the configuration may be made such that the main lumen S1 and the expansion lumens S2 and S2a do not extend to the proximal ends of the proximal shaft portions 16 and 16a (shafts 10 and 10a) but bend halfway and open to the outer peripheral surface of the shaft 10. Further, the expansion lumens S2 and S2a may be configured to extend to the distal shaft portions 14 and 14a. In the above-mentioned embodiments, the number of the lumens formed in the distal shaft portions 14 and 14a may be 2 or more, and the number of the lumens formed in the proximal shaft portions 16 and 16a may be 3 or more. In essence, in the configuration in which the first shaft portion and the second shaft portion are formed of the same material, the first shaft portion is formed in a shape having a cross section more flexible (i.e., less rigid in bending) than that of the second shaft portion, so that it becomes possible to achieve the shaft in the scope of claims. For example, in the above-mentioned embodiments, the configuration is made such that the number of the lumens formed in the first shaft portion is less than the number of the lumens formed in the second shaft portion, and the outer diameter of the first shaft portion is smaller than the outer diameter of the second shaft portion. Further, the shaft may be configured such that the first shaft portion is formed of a material having hardness lower than that of the material forming the second shaft portion.

In the above-mentioned embodiments, the distal portion 32 of the balloon 30 may be configured to join to the slightly further proximal side than the distal portions of the proximal shaft portions 16 and 16a. That is, the balloon 30 may be configured to join to the distal side of the proximal shaft portions 16 and 16a. In essence, the balloon catheter in the scope of claims only needs to have a configuration in which the first shaft portion having relatively high flexibility is disposed on the further distal side than the second shaft portion to which the balloon is joined.

In the above-mentioned embodiments, the balloon catheters 100 and 100a may be configured without the hydrophilic coatings 18 and 18a. Further, in the above-described first embodiment, the configuration may be made such that a side hole is formed in the outer peripheral surface of the distal shaft portion 14. In the above-described second embodiment, the configuration may be made such that no side hole is formed in the outer peripheral surface of the distal shaft portion 14a.

In the above-mentioned embodiments, the configuration may be made such that the outer diameter of the shafts 10 and 10a are substantially the same over the entire length. Further, the shape of the cross section of the shafts 10 and 10a is not limited to a circular shape and may be a polygonal shape or the like. Further, the entire shafts 10 and 10a, the distal shaft portions 14 and 14a, or the proximal shaft portions 16 and 16a are not limited to a single-layer structure, and they may be configured to include a plurality of resin layers (e.g., an inner layer resin and an outer layer resin) or have a structure in which a reinforcing body (blade), a coil body, or the like is further embedded. In the above-mentioned embodiments, when the shafts 10 and 10a include a plurality of the resin layers and the reinforcing body, the configuration may be made such that, for each of the plurality of the resin layers and the reinforcing body, the distal shaft portions 14 and 14a and the proximal shaft portions 16 and 16a are integrally formed using the same material.

Further, the material of each member in the above-mentioned embodiments is merely an example, and various modifications are possible.

In the above-mentioned embodiments, the balloon catheters 100 and 100a to be inserted into the blood vessel are exemplified as the balloon catheter. However, the balloon catheter may be the one to be inserted into a body lumen other than the blood vessel (e.g., the esophagus, etc.).

### DESCRIPTION OF REFERENCE NUMERALS

### 10, 10a: Shaft

### 12: Distal tip

14, 14a: Distal shaft portion
16, 16a: Proximal shaft portion
18, 18a: Hydrophilic coating
20: Side hole

### 30: Balloon

32: Distal portion
34: Rear portion

### 40: Connector

100, 100a: Balloon catheter
B1: External carotid artery
B2: Cervical part of internal carotid artery
B3: Petrous part of internal carotid artery
B4: Ophthalmic artery
B5: Anterior cerebral artery
B6: Middle cerebral artery
B: Common carotid artery
C: Thrombus piece

### P: Petrous

S1: Main lumen
S2, S2a: Expansion lumen
S3: Internal space

1. A balloon catheter comprising:
a shaft including a first shaft portion and a second shaft portion located on a proximal side of the first shaft portion; and
a balloon covering and joined to the second shaft portion, wherein the first shaft portion is more flexible than the second shaft portion and has a length in an axial direction of 1.5 cm or more.

2. The balloon catheter according to claim 1, wherein:
an outer layer of the first shaft portion and an outer layer of the second shaft portion are each formed of a resin; and
a resin material of the outer layer of the first shaft portion has a Shore hardness of D47 or less.

3. The balloon catheter according to claim 1, wherein:
an outer layer of the first shaft portion and an outer layer of the second shaft portion are each formed of a resin; and
a resin material of the outer layer of the first shaft portion has a Shore hardness of D40 or less.

4. The balloon catheter according to claim 1, wherein:
a first lumen penetrating from a proximal side of the second shaft portion to a distal end of the first shaft portion is formed in the shaft; and
a side hole communicating with the first lumen is formed in an outer peripheral surface of the first shaft portion on a proximal side.

5. The balloon catheter according to claim 1, wherein a hydrophilic coating is formed on at least a distal side of an outer peripheral surface of the first shaft portion.

6. The balloon catheter according to claim 5, wherein:
the hydrophilic coating is formed on the outer peripheral surface of a distal portion of the first shaft portion; and
a coefficient of friction of the outer peripheral surface of the first shaft portion at a position proximal of the hydrophilic coating is higher than a coefficient of friction of the hydrophilic coating.

7. The balloon catheter according to claim 1, wherein:
the first shaft portion and the second shaft portion are integrally formed;
a first lumen and a second lumen are formed in the shaft, the first lumen penetrating from a proximal side of the second shaft portion to a distal end of the first shaft portion, the second lumen extending from the proximal side of the second shaft portion to an inside of the balloon and communicating with the balloon; and
an outer diameter of the first shaft portion is smaller than an outer diameter of the second shaft portion.
